(19) United States
(12) Patent Application Publication
(10) Pub. No.: US 2019/0269352 A1
(43) Pub. Date: Sep. 5, 2019

(54) PROVIDING CORRECTIVE FEEDBACK FOR GAIT MODIFICATION

(71) Applicant: International Business Machines Corporation, Armonk, NY (US)

(72) Inventors: Douglas S. Brown, Charlotte, NC (US); John F. Kelley, Polk City, FL (US); Todd P. Seager, Orem, UT (US); Helaine M. Wasser, Cambridge, MA (US)

(21) Appl. No.: 15/911,464

(22) Filed: Mar. 5, 2018

(51) Int. Cl. A61B 5/103; A61B 5/11; G06F 3/01; A43B 3/00

(52) U.S. Cl. CPC A61B 5/1038; A61B 5/112; A43B 3/0005; G06F 3/016; A61B 5/1116

(57) ABSTRACT

A user's gait or posture may require modification for numerous reasons, such as medical conditions. Further, gait correction may also provide benefits for participation in sporting events or while exercising. An embodiment of the present invention includes a system that provides corrective feedback for gait modification. The system uses one or more pressure sensors to measure pressure applied by a user. A stimulation device applies a stimulation to one or more portions of a user's foot to inform the user to alter pressure applied to those portions of the foot in order to adjust the user's gait or posture. Embodiments of the present invention further include a method for providing corrective feedback in substantially the same manner described above.

### BACKGROUND

### 1. Technical Field

Present invention embodiments relate to systems providing corrective feedback, and more specifically, to a system that provides corrective feedback for gait modification. A user's gait or posture may require modification for numerous reasons, such as medical conditions. Further, gait correction may also provide benefits for participation in sporting events or while exercising. Embodiments of the present invention collect gait information and, based on the information collected, apply corrective stimuli to the user to encourage gait modification.

### 2. Discussion of the Related Art

Human gait refers to bipedal locomotion such as walking, jogging, running, and sprinting. A gait cycle can be divided into two general phases, stance phase and swing phase. Stance phase is the part of a gait cycle during which a reference foot remains in contact with the ground, and swing phase occurs when the reference foot is not in contact with the ground. An individual's gait may be characterized by differences in limb movement patterns, overall velocity, forces, kinetic and potential energy cycles, and changes in the contact with the ground. People may consciously change their gait for a variety of reasons. For example, the gait of someone who is sprinting may not involve a heel strike, unlike the gait of an individual who is jogging.

An individual may wish to modify his or her gait in order to ambulate more effectively and safely. Illness or injuries may negatively affect the gait, leaving pathologies that can be corrected by therapy. Similarly, athletes may find that changes to their gait can provide a competitive advantage. Modifying one's gait may require expert coaching and hours of practice before new behaviors are adopted.

### SUMMARY

According to one embodiment of the present invention, a system provides corrective feedback for gait modification. The system uses one or more pressure sensors to measure pressure applied by a user. A stimulation device applies a stimulation to one or more portions of a user's foot to inform the user to alter pressure applied to those portions of the foot in order to adjust the user's gait or posture. Embodiments of the present invention further include a method for providing corrective feedback in substantially the same manner described above.

### BRIEF DESCRIPTION OF THE DRAWINGS

Generally, like reference numerals in the various figures are utilized to designate like components.

FIG. 1 is a block diagram depicting a computing environment for a system providing corrective feedback in accordance with an embodiment of the present invention;

FIG. 2A is a view in perspective of a footwear item of a system providing corrective feedback in accordance with an embodiment of the present invention;

FIG. 2B is a bottom view in plan of a footwear item of a system providing corrective feedback in accordance with an embodiment of the present invention;

FIG. 2C is a top view in perspective of a footwear item of a system providing corrective feedback in accordance with an embodiment of the present invention;

FIG. 2D is an exploded view in perspective of a footwear item of a system providing corrective feedback in the form of a ski boot coupled to a ski binding in accordance with an embodiment of the present invention;

FIG. 3 is a flow chart depicting operations for providing corrective feedback in accordance with an embodiment of the present invention;

FIG. 4 is a flow chart depicting operations for determining corrective feedback in accordance with an embodiment of the present invention;

FIG. 5 is a flow chart depicting operations for releasing a ski binding in accordance with an embodiment of the present invention; and

FIG. 6 is a block diagram depicting a computing device, in accordance with an embodiment of the present invention.

### DETAILED DESCRIPTION

Present invention embodiments relate generally to systems providing corrective feedback, and more specifically, to a system that provides corrective feedback to encourage a user to modify the user's gait or posture. A user's gait or posture may require modification for numerous reasons. Medical conditions, such as stroke and plantar fasciitis, may hamper the user's ability to walk properly. Gait correction may also provide benefits for participation in sporting events or while exercising. Effective, injury-free walking and running may involve training to achieve an optimal movement and placement of the foot. Unfortunately, coaching techniques may require experts and can be time-consuming, since lessons must be understood, remembered, and practiced consistently. Embodiments of the present invention collect gait information using a system including a sensor-equipped footwear item. Based on the information collected, the system then applies corrective stimuli to the user to encourage gait modification.

It should be noted that references throughout this specification to features, advantages, or similar language herein do not imply that all of the features and advantages that may be realized with the embodiments disclosed herein should be, or are in, any single embodiment of the invention. Rather, language referring to the features and advantages is understood to mean that a specific feature, advantage, or characteristic described in connection with an embodiment is included in at least one embodiment of the present invention. Thus, discussion of the features, advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages will become more fully apparent from the following drawings, description and appended claims, or may be learned by the practice of the invention as set forth hereinafter.

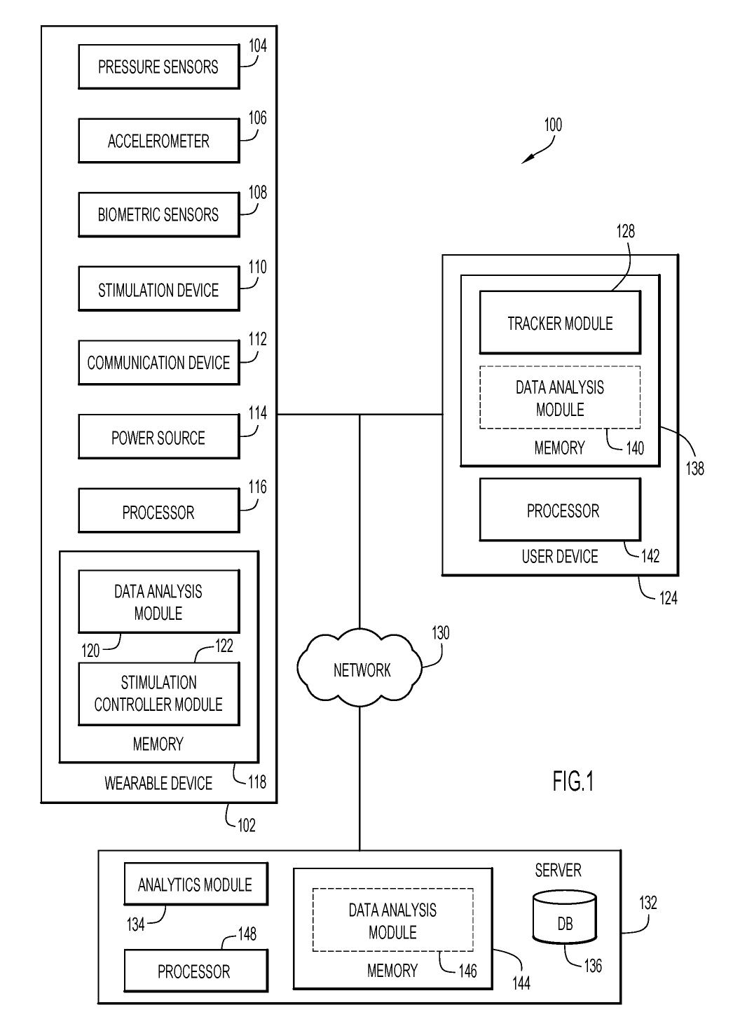

Present invention embodiments will now be described in detail with reference to the Figures. FIG. 1 is a block diagram depicting a computing environment 100 for a system providing corrective feedback in accordance with an embodiment of the present invention. As depicted, computing environment 100 includes a footwear item or wearable device 102, a user device 124, and a server 132. Footwear item 102 includes pressure sensors 104, accelerometer 106, biometric sensors 108, a stimulation device 110, a communication device 112, a power source 114, a processor 116, and a memory 118 with a data analysis module 120 and a stimulation controller module 122. User device 124 includes memory 138 with tracker module 128. Server 132 includes an analytics module 134 and a database 136. It is to be understood that the functional division among components of computing environment 100 have been chosen for purposes of explaining embodiments of the present invention and is not to be construed as a limiting example.

Footwear item 102 may be any item worn on the person that may equipped with a variety of sensors and modules. Footwear item 102 preferably includes a sock, socklet, shoe, slipper, nylons, hosiery, or any other item worn on or associated with the foot. In some embodiments, footwear item 102 is worn directly on the foot, and a shoe, sneaker, boot, etc. is able to be worn over footwear item 102. Footwear item 102 may include electronic textiles, also known as e-textiles or smart fabrics, which are woven into footwear item 102 and may enable sharing of information between the elements of footwear item 102. Elements of footwear item 102 may be detachable or removable. In one embodiment, some elements of footwear item 102 are removable prior to laundering or cleaning footwear item 102. Footwear item 102 may be composed of materials such as cotton, wool, nylon, linen, cashmere, mohair, silk, acrylic, polyester, olefins, spandex, or any other material (or combination of materials) suitable for a footwear item.

Pressure sensors 104 may include one or more sensors or transducers used to measure pressures and/or forces acting on footwear item 102. Pressure sensors 104 may be placed throughout footwear item 102 in order to collect pressure data from different regions of the foot. Pressure sensors 104 may be placed in an area corresponding to the first metatarsophalangeal joint (e.g., the ball of the foot), the fifth metatarsophalangeal joint, the medial side of the heel, and the lateral side of the heel. In some embodiments, pressure sensors 104 are located anywhere on footwear item 102 that would enable pressure sensors 104 to collect pressure data of interest. Thus, pressure sensors 104 may be placed in any location suitable for the collection of pressure data relating to a user's gait and/or posture.

Accelerometer 106 may include any sensor that measures acceleration in one or more dimensions. One or more accelerometers 106 may be placed throughout footwear item 102 in order to collect information of interest regarding a user's gait and/or posture. By collecting both pressure and acceleration data, a user's gait may be profiled in order to provide customized gait-correcting feedback.

Biometric sensors 108 may include any sensors capable of collecting and measuring biosignals. Biosignals are any electrical or non-electrical signals in living organisms that can be measured and monitored. Biometric sensor 108 may include one or more of a heart rate monitor, blood pressure monitor, temperature monitor, and pulse oximeter. Thus, biometric sensors 108 can measure a user's heart rate, blood pressure (systolic and diastolic), temperature, and oxygen saturation. Each biometric sensor 108 may be placed on a location of footwear item 102 that is conducive to the collection of its data; for example, a temperature sensor may be positioned on the inside of footwear item 102 so that it may come into contact with the user's skin.

Stimulation device 110 may include any device capable of stimulating a user of footwear item 102. Stimulation device 110 may stimulate parts of a user's body in order to provide feedback that corrects the user's gait and/or posture. In one embodiment, stimulation device 110 uses transcutaneous electrical nerve stimulation (TENS) or electrical muscle stimulation (EMS). By applying an electrical current to particular portions of the foot, stimulation device 110 reminds the user to avoid placing pressure on that portion of the foot. Stimulation device 110 may elicit particular muscle contractions using electric impulses. In one embodiment, stimulation device 110 includes a vibration device (such as a linear resonant actuator or eccentric rotating mass motor) to transmit haptic feedback to the user's foot. Thus, stimulation device 110 may mimic a sensation akin to a “rock in a shoe” to encourage a user to adjust his or her gait and/or posture accordingly.

Communication device 112 may include any wired or wireless network interface or communications devices (e.g., modem, network card). Communication device 112 may transmit data collected by sensors of footwear item 102 to user device 124 or server 132 via a network 130. Footwear item 102 may also receive data from server 132 and user device 124 using communication device 112.

Power source 114 may provide footwear item 102 with electrical signals to power various components, such as the sensors 104-108, stimulation device 110, communication device 112, processor 116, and memory 118. Power source 114 may be an electrical battery, such as an alkaline battery or lithium-ion battery. Power source 114 may be removable so that a user can remove a battery (e.g., if the battery is drained, or in order to wash footwear item 102). In one embodiment, power source 114 is recharged by converting a user's kinetic energy to potential energy as a user moves. Power source 114 may be located in an area of footwear item 102 that does not experience a substantial amount of pressure from a user.

Data analysis module 120 and stimulation controller module 122 may include one or more modules or units to perform various functions of present invention embodiments described below. The modules may be implemented by any combination of any quantity of software and/or hardware modules or units, and may reside within memory 118 of footwear item 102, in memory 138 of user device 124 (e.g., data analysis module 140) for execution by a corresponding processer, such as processor 142, and/or in memory 144 on server 132 (e.g., data analysis module 146) for execution by a corresponding processor, such as processor 148.

Data analysis module 120 may receive and analyze data from sensors, such as pressure sensors 104, accelerometer 106, and biometric sensor 108. For example, data analysis module 120 may detect abnormalities or irregularities in a user's gait and/or posture by analyzing the pressure data to detect pressure events (e.g., steps), the time that the events occur, the acceleration forces experienced, and other biometric information (such as a user's pulse, oxygen saturation, temperature, blood pressure, etc.). Data analysis module 120 may also factor in information provided by a user, such as height, weight, in-seam length, stride length, shoe size, body-mass index (BMI), age, and gender. After analyzing the data, data analysis module 120 may determine stimulation instructions that are intended to correct a user's gait and/or posture.

Stimulation controller module 122 may control stimulation device 110 by interpreting stimulation instructions from data analysis module 120 to provide stimuli to a user via stimulation device 110. For example, if data analysis module 120 determines that a user suffers from over-pronation or over-supination, then data analysis module 120 may determine that certain parts of the user's foot should be stimulated in order to bring a user's attention to a misaligned foot placement. Stimulation controller module 122 may take this information and determine which parts of the foot should be stimulated, as well as the timing of the stimulation. At the appropriate times, stimulation controller module 122 directs stimulation device 110 to stimulate those areas of the foot. In some embodiments, data analysis module 120 and stimulation controller module 122 function together to provide corrective stimulations that adjust on-the-fly to changes in a user's gait and/or posture.

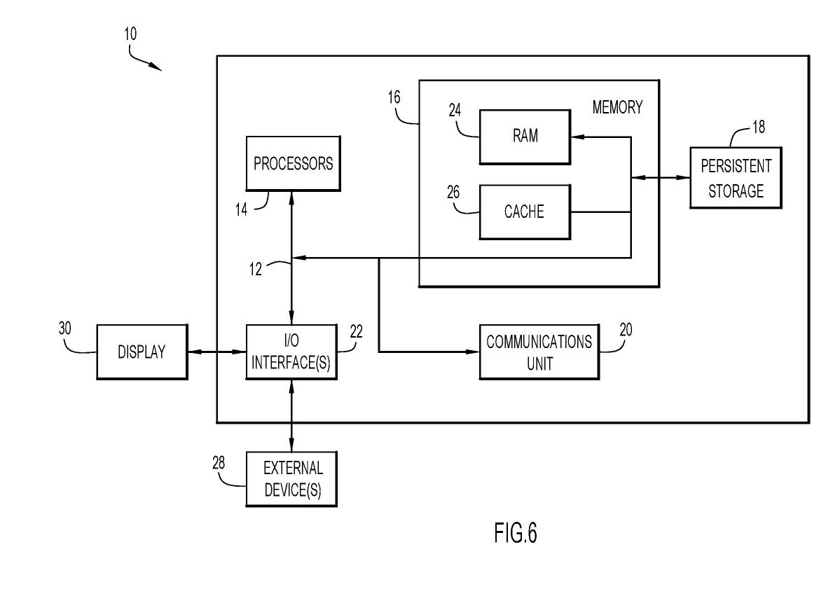

User device 124 may include any device by which a user may interact with footwear item 102. In various embodiments of the present invention, user device 102 may be a laptop computer, a tablet computer, a netbook computer, a personal computer (PC), a desktop computer, a personal digital assistant (PDA), a smart phone, a thin client, or any programmable electronic device capable of executing computer readable program instructions. User device 124 may include internal and external hardware components, as depicted and described in further detail with respect to FIG. 6. User device 124 may enable a user to link footwear item 102 with a health-related software application to provide accurate measurements of walking/jogging activity, diagnostics for correcting gait, and the like. In some embodiments, data analysis module 140 resides on user device 124; sensor data is analyzed on user device 124, and stimulation instructions are sent back to stimulation controller module 122 of footwear item 102.

User device 124 may also track a user's various health metrics with tracker module 128. For example, tracker module 128 may collect and store any data collected by pressure sensors 104, accelerometer 106, biometric sensors 108, as well as the results of data analysis module 120 and any data relating to the stimuli applied by stimulation device 110. Thus, tracker module 128 may keep a historical record corresponding to usage of footwear item 102, which may enable a user to track aspects of his or her gait and/or posture history (e.g., changes in gait and/or posture over time).

Network 130 can be, for example, a local area network (LAN), a wide area network (WAN) such as the Internet, or a combination of the two, and include wired, wireless, or fiber optic connections. In general, network 130 can be any combination of connections and protocols that will support communications between footwear item 102, user device 124, and/or server 132 in accordance with an embodiment of the present invention.

Server 132 may receive any data from footwear item 102 and/or user device 124 regarding the gait of the user, corrective stimuli applied, user health metrics (either collected from biometric sensor 108 or supplied by the user), user activity, and the like. Analytics module 134 may analyze the collected data in order to refine the algorithms used for providing corrective feedback. For example, if analytics module 134 determines that users sharing a common gait and/or posture problem respond best to a stimulation of a certain magnitude and/or duration, analytics module 134 may suggest or provide an updated algorithm that varies the stimulation magnitude and/or duration of stimulation accordingly. Analytics module 134 may take into account various user metrics that are collected while the footwear item is worn, such as a user's blood oxygenation, temperature, pulse, heartrate, stride length, and the like. Thus, analytics module 134 may use analytic approaches to continually refine corrective feedback algorithms used by footwear item 102. In some embodiments, server 132 may send updated or improved algorithms or analytics to one or more modules of footwear item 102 (such as data analysis module 120 or stimulation controller 122).

Database 136 may be any non-volatile storage media known in the art. For example, database 136 can be implemented with a tape library, optical library, one or more independent hard disk drives, or multiple hard disk drives in a redundant array of independent disks (RAID). Similarly, data on database 136 may conform to any suitable storage architecture known in the art, such as a file, a relational database, an object-oriented database, and/or one or more tables. Server 132 may store data collected from each footwear item 102 that is used by analytics module 134, as well as output of analytics module 134.

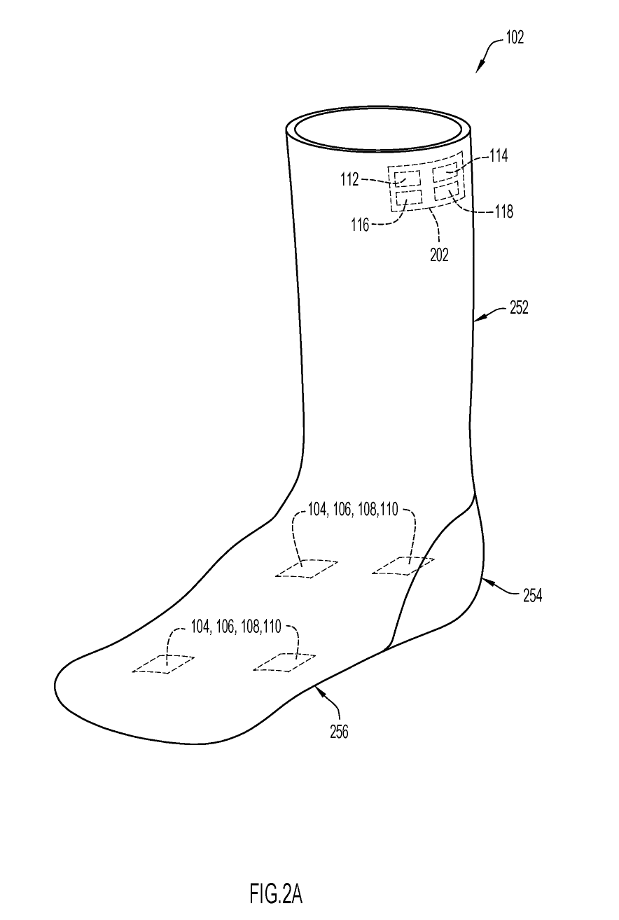

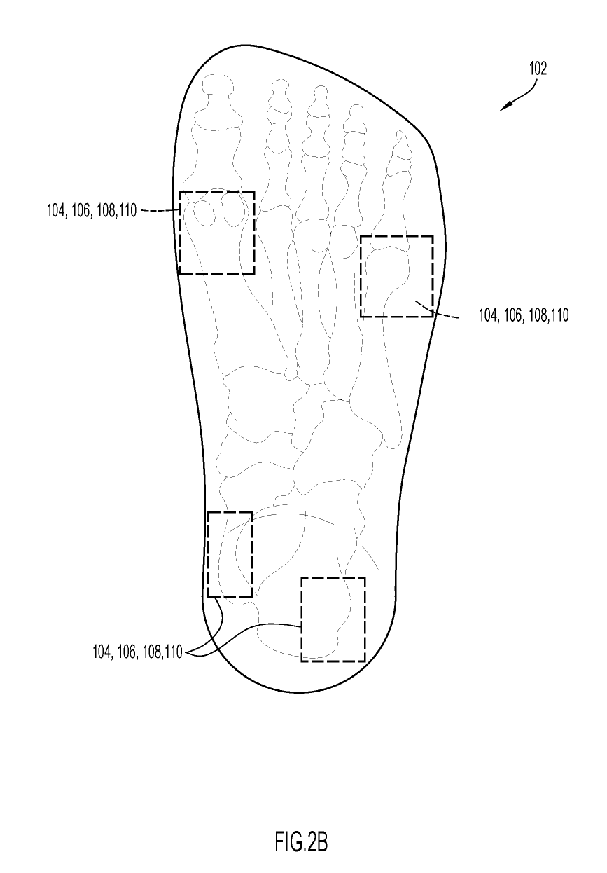

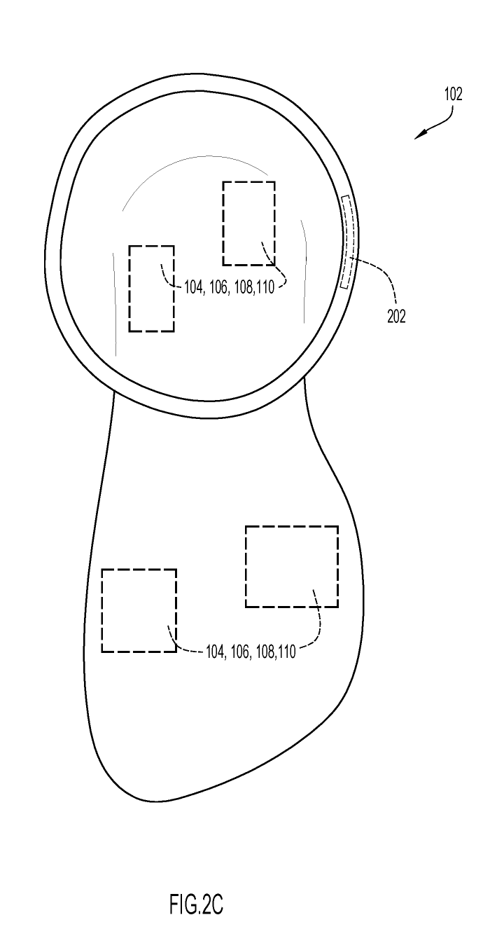

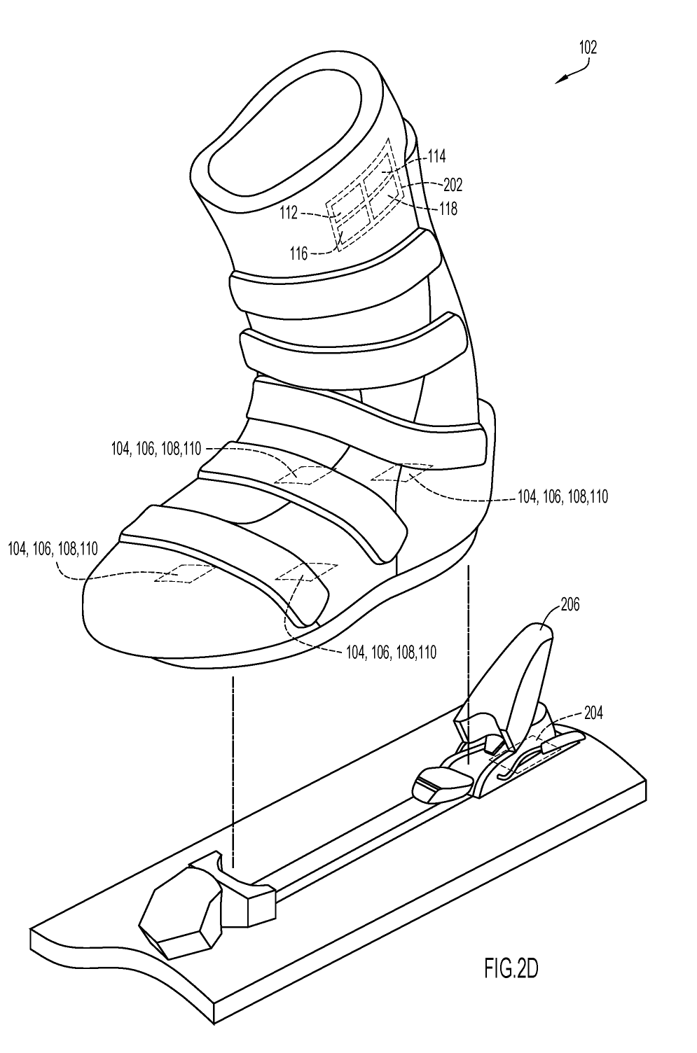

FIGS. 2A-2D are schematic representations depicting an example footwear item 102 of a system providing corrective feedback in accordance with an embodiment of the present invention. FIG. 2A is a perspective view of footwear item 102, FIG. 2B is a bottom view in plan, and FIG. 2C is a top view in perspective. As depicted, footwear item 102 may include pressure sensors 104, accelerometers 106, biometric sensors 108, stimulation devices 110, and enclosure 202 that encloses communications device 112, power source 114, processor 116, and memory 118. The depicted embodiment depicts footwear item 102 having a shape that makes it is suitable to be worn on a user's left foot; in other embodiments, footwear item 102 may have a shape that enables a user to wear footwear item 102 on a right foot, or footwear item 102 may be capable of being worn on either foot.

Footwear item 102 may have a generally “L”-shaped body, including a proximal section 252 corresponding to a cuff, a distal section 256 for covering the foot, and an intermediate section 254 for encompassing a heel of the foot. Footwear item 102 may include one or more pressure sensors 104 placed in locations corresponding to areas of interest of a user's foot. For example, pressure sensors 104 may be placed at the first metatarsophalangeal joint (e.g., the ball of the foot) and at the fifth metatarsophalangeal joint. A pressure sensor 104 under and/or along the medial side under the first metatarsophalangeal joint may detect pressures during stages of gait, such as the last stage of gait (toe-off), or when a user is sprinting on the toes. A pressure sensor 104 may be placed in a location under and/or along the lateral side of the fifth metatarsophalangeal joint to detect pressures experienced during gait, such as the last stage of gait (toe-off). A pressure sensor 104 placed along the medial and lateral sides of the foot may be useful for detecting conditions such as over-pronation or over-supination. A pressure sensor 104 placed along the lateral aspect of the calcaneus (i.e., lateral of the calcaneus in plantar view) and the medial aspect of the calcaneus (i.e., medial of the calcaneus in plantar view) area may detect pressures associated with the heel, such as those experienced during the heel strike stage of gait.

Accelerometers 106, biometric sensors 108, and stimulation devices 110 may be located near each pressure sensor 104. In some embodiments, placement of accelerometers 106, biometric sensors 108, and/or stimulation devices 110 may be independent of pressure sensors 104. Stimulation device 110 may be positioned on top of the foot so that stimulation device 110 can provide the user with feedback unrelated to the user's gait; for example, stimulation device 110 may stimulate the top of a user's foot generally each time the user should be stepping on that foot in order to encourage a particular step rate.

Communication device 112, power source 114, processor 116, and memory 118 may be located in the leg or cuff of footwear item 102. Communication device 112, power source 114, processor 116, and memory 118 may be enclosed in enclosure 202, which may provide protection against water, light, sweat, and/or temperature extremes. In some embodiments, communications device 112, power source 114, processor 116, and/or memory 118 are located on footwear item 102 in a location that would be outside of the rim or collar of a shoe, enabling footwear item 102 to be worn with a shoe such that enclosure 202 does not come into contact with the shoe. Communications device 112, power source 114, processor 116, and memory 118 may be located in proximity to each other, or spread throughout the leg or cuff of footwear item 102. In some embodiments, communications device 112, power source 114, processor 116, and/or memory 118 may be located in the other portions of footwear item 102, such as the dorsal aspect (top) of the foot, including the metatarsals, and the medial or lateral malleolus (ankle).

FIG. 2D depicts an exploded view in perspective of an example ski boot that couples to a binding 206 with one or more release mechanisms 204 (e.g., manual lever, electronically controlled, etc.). Footwear item 102 (e.g., FIGS. 2A-2C) may be worn by a user on the foot/leg and inserted inside of a ski boot. Alternatively, footwear item 102 may be a ski boot itself, with sensors and power and processing components disposed on the ski boot at locations corresponding to the locations described above for footwear item 102 of FIGS. 2A-2C. Footwear item 102 may wirelessly communicate release instructions to release mechanisms 204 in order to prevent an injury to a user. For example, if footwear item 102 experiences excessive lateral or twisting motion (as measured by accelerometer 106), then footwear item 102 may trigger release mechanism 204 to activate, thus freeing a skier's boot or foot from the ski binding 206. The release mechanism 204 may release the binding 206 using a magnetically-controlled fastener, a pyrotechnic fastener, solenoid, or other release mechanisms.

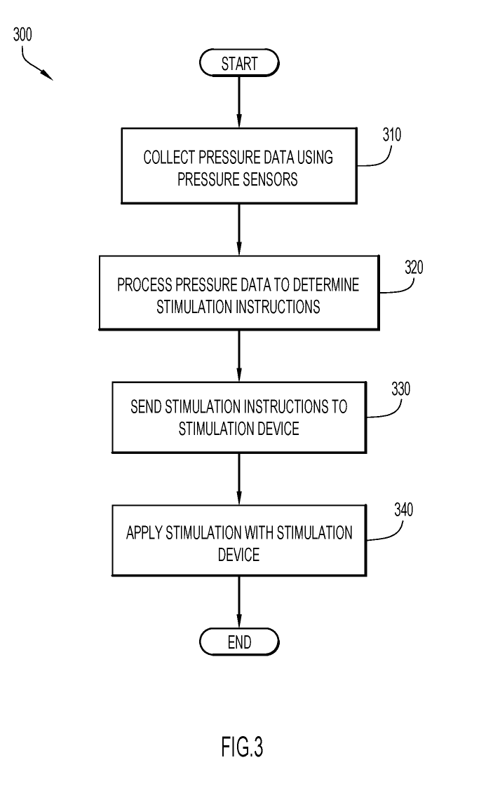

FIG. 3 is a flow chart depicting a method 300 for providing corrective feedback in accordance with an embodiment of the present invention.

Pressure data is collected using pressure sensors at operation 310. In some embodiments, pressure data is collected by one or more pressure sensors 104. Pressure data may include any forces or pressures experienced by footwear item 102 as a user moves or stands. As a user proceeds through the phases of gait (e.g., initial contact (heel strike), loading response (foot flat), mid-stance, terminal stance, toe off/pre-swing), various regions of footwear item 102 will experience different pressures. For example, when a user is walking heel-to-toe, the heel contacts the ground first, so pressure measured at a location of footwear item 102 corresponding to the toe would be near-zero. By measuring pressure experienced on different regions of a user's foot, a user's gait and posture can be analyzed and abnormalities can be diagnosed.

Pressure data is processed at operation 320 to determine stimulation instructions. In some embodiments, data analysis module 120 analyzes data from pressure sensors 104 in order to identify gait and/or posture problems that can be corrected. Data analysis module may then output stimulation instructions that may correct the identified problems. In general, data analysis module 120 may identify areas of the foot that may be applying too much pressure, and direct stimulation to those areas in order to discourage the user from shifting too much weight there. For example, pressure measurements may be compared to profiles of pressure measurements for appropriate gait and/or posture to determine areas of the foot applying excess or insufficient amounts of pressure. Some or all of the processing of pressure data may take place on user device 124 or server 132 (e.g., by data analysis module 140 or data analysis module 146).

Stimulation instructions are sent to a stimulation device at operation 330. Stimulation instructions may include such details as type of stimulation (e.g., electrical, mechanical/haptic), duration of stimulation, and magnitude of stimulation. Stimulation instructions may repeat or loop in order to encourage a pattern of gait and/or posture, or may be re-calculated every time a user takes a step.

Stimulation is applied with the stimulation device at operation 340. Stimulation may be applied with stimulation device 110 according to the stimulation instructions interpreted by stimulation controller module 122 that are received in operation 330. Stimulation device 110 may apply stimulation to one or multiple areas of a user's foot at any given time in order to encourage proper gait or posture.

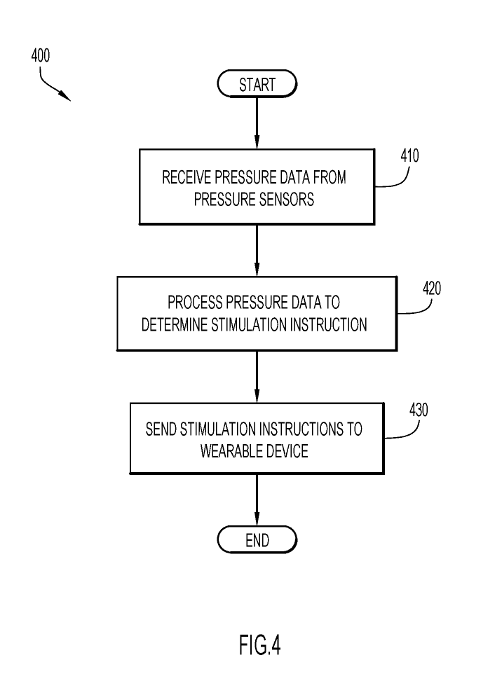

FIG. 4 is a flow chart depicting a method 400 for determining corrective feedback in accordance with an embodiment of the present invention.

Pressure data is received from pressure sensors at operation 410. In some embodiments, pressure data is received by server 132 or user device 124 from footwear item 102 via network 130.

Pressure data is processed to determine stimulation instructions at operation 420. The pressure data may be processed by user device 124 or server 132 in order to determine the appropriate stimuli to apply to the user. A copy of the pressure data or stimulation instructions may be retained by server 132 in database 136. In some embodiments, pressure data is processed to determine stimulation instructions that would correct a user's gait and/or posture. Pressure data may be processed to determine stimulation instructions intended to improve a user's performance in areas such as track and field events, basketball, gymnastics, soccer, long-jumping, mountain climbing, driving a motor vehicle, dressage, and any other event involving foot placement, movement, and/or footfall timing.

Stimulation instructions are sent to the footwear item at operation 430. The stimulations instructions may be sent to footwear item 102 from the processing location of operation 420 (e.g., user device 124 or server 132). The stimulation instructions may describe stimuli location, duration, and magnitude in a form that is readable by stimulation controller module 122 of footwear item 102. These may be based on comparisons of the received pressure data with the profiles. For example, an area receiving excessive pressure may have a greater stimulation for a longer time interval.

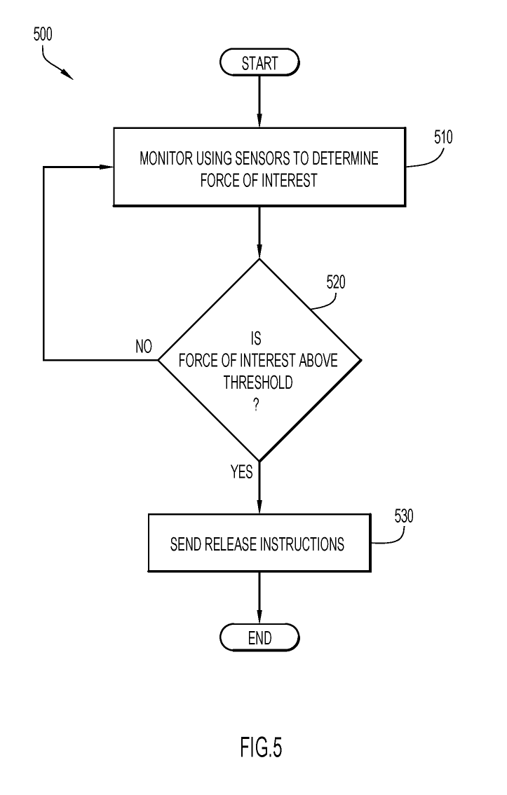

FIG. 5 is a flow chart depicting a method 500 for releasing a ski binding in accordance with an embodiment of the present invention. Bindings may refer to any device that binds a foot or shoe. In one embodiment, the binding is the ski binding 206 (see FIG. 2D), which connect a ski boot to a ski. The footwear item 102 of environment 100 may be worn between a user's foot and a ski boot.

Sensors are monitored to determine forces of interest at operation 510. These forces of interest may include pressure or torque relating to a user's foot. The forces of interest may be selected based on measurable forces that may cause injury to a skier; for example, lateral or twisting forces.

When a sensor measures a force of interest, that force may be compared to a threshold at operation 520. In some embodiments, each force that a sensor measures is compared to a threshold; in other embodiments, a running average of force over time is computed and compared. In some embodiments, the force of interest is compared to a threshold to detect a sudden stop, change in acceleration (jerk), or change in momentum. If the force exceeds a threshold, then the method 500 may proceed to operation 530; otherwise, the method 500 may loop back to operation 510.

Release instructions are sent at operation 530. These release instructions may instruct the binding to release the boot or foot of the user. In some embodiments, the footwear item 102 wireless transmits release instructions to the release mechanism 204 using communications device 112.

FIG. 6 is a block diagram depicting components of a computer 10 suitable for executing the methods disclosed herein. Computer 10 may implement processors of footwear item 102, and/or other processing and memory components of user device 124, and/or server 132 to provide corrective feedback in accordance with embodiments of the present invention. It should be appreciated that FIG. 6 provides only an illustration of one embodiment and does not imply any limitations with regard to the environments in which different embodiments may be implemented. Many modifications to the depicted environment may be made.

As depicted, the computer 10 includes communications fabric 12, which provides communications between computer processor(s) 14, memory 16, persistent storage 18, communications unit 20, and input/output (I/O) interface(s) 22. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses.

Memory 16 and persistent storage 18 are computer readable storage media. In the depicted embodiment, memory 16 includes random access memory (RAM) 24 and cache memory 26. In general, memory 16 can include any suitable volatile or non-volatile computer readable storage media.

One or more programs may be stored in persistent storage 18 for execution by one or more of the respective computer processors 14 via one or more memories of memory 16. The persistent storage 18 may be a magnetic hard disk drive, a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Communications unit 20, in these examples, provides for communications with other data processing systems or devices. In these examples, communications unit 20 includes one or more network interface cards. Communications unit 20 may provide communications through the use of either or both physical and wireless communications links.

I/O interface(s) 22 allows for input and output of data with other devices that may be connected to computer 10. For example, I/O interface 22 may provide a connection to external devices 28 such as a keyboard, keypad, a touch screen, and/or some other suitable input device. External devices 28 can also include portable computer readable storage media such as, for example, thumb drives, portable optical or magnetic disks, and memory cards.

Software and data used to practice embodiments of the present invention can be stored on such portable computer readable storage media and can be loaded onto persistent storage 18 via I/O interface(s) 22. I/O interface(s) 22 may also connect to a display 30. Display 30 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The footwear item information, user information, and stimulation information may be stored within any conventional or other data structures (e.g., files, arrays, lists, stacks, queues, records, etc.) and may be stored in any desired storage unit (e.g., database, data or other repositories, queue, etc.) The footwear item information, user information, and stimulation information transmitted between footwear item 102, user device 124, and server 132 may include any desired format and arrangement, and may include any quantity of any types of fields of any size to store the data. The definition and data model for the footwear item information, user information, or stimulation information, or messages containing the footwear item information, user information, or stimulation information, may indicate the overall structure in any desired fashion (e.g., computer-related languages, graphical representation, listing, etc.).

Footwear item information may include any information collected by footwear item 102, any information derived by analyzing information collected by footwear item 102, and any information otherwise provided to footwear item 102, user device 124, and server 132. Footwear item information may include any measurable information collected from a user by any collection mechanism (e.g., sensor, transducer, etc.), any combination of measurable information, and any information derived from analyzing collected information. User information may include any information relating to a user of the footwear item, either collected by the footwear item, supplied by the user, or provided from any other source. Stimulation information may include any information relating to the stimuli provided to a user of footwear item, such as location of stimuli, duration of stimuli, magnitude of stimuli, type of stimuli applied (e.g., mechanical, haptic, electrical, aural, etc.), and timing of stimuli (e.g., stimulation patterns, rhythms, or tempos). Footwear item information, user information, and stimulation information may include any desired format and arrangement, and may include any quantity of any types of fields of any size to store any desired data. The fields may indicate the presence, absence, actual values, or any other desired characteristics of the data of interest (e.g., quantity, value ranges, etc.). Footwear item information, user information, and stimulation information may include all or any desired portion (e.g., any quantity of specific fields) of personal information (PI) or other data of interest within a given implementation or system.

The present invention embodiments may employ any number of any type of user interface (e.g., Graphical User Interface (GUI), command-line, prompt, etc.) for obtaining or providing information (e.g., user information and metadata), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The present invention embodiments are not limited to the specific tasks or algorithms described above, but may be utilized for providing feedback to a user for various types of endeavors, including any event or activity where a user's foot or feet may be involved.

It will be appreciated that the embodiments described above and illustrated in the drawings represent only a few of the many ways of implementing embodiments for providing corrective feedback with footwear items.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., communications software, client software, server software, tracker module, data analysis module, stimulation controller module, and analytics module, etc.). These systems may include any types of monitors and input devices (e.g., keyboard, mouse, voice recognition, etc.) to enter and/or view information.

It is to be understood that the software (e.g., communications software, client software, server software, tracker module, data analysis module, stimulation controller module, and analytics module) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., tracker module 128, data analysis module 120, stimulation controller module 122, and analytics module 134) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., pressure data, acceleration data, biometric data, stimulation instructions, gait information). The database system may be implemented by any number of any conventional or other databases, data stores or storage structures (e.g., files, databases, data structures, data or other repositories, etc.) to store information (e.g., pressure data, acceleration data, biometric data, stimulation instructions, gait/posture information, etc.). The database system may be included within or coupled to the server and/or client systems. The database systems and/or storage structures may be remote from or local to the computer or other processing systems, and may store any desired data (e.g., pressure data, acceleration data, biometric data, stimulation instructions, gait/posture information, etc.).

The present invention embodiments may employ any number of any type of user interface (e.g., Graphical User Interface (GUI), command-line, prompt, etc.) for obtaining or providing information (e.g., pressure data, acceleration data, biometric data, stimulation instructions, gait/posture information, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. An apparatus comprising:
a footwear item comprising:
one or more pressure sensors to measure pressure applied by a user; and
a stimulation device to apply stimulation to one or more portions of a foot to inform the user to alter pressure applied to those portions of the foot to adjust one or more from a group of a gait and a posture of the user.

2. The apparatus of claim 1, further comprising:
a processing device to analyze the measured pressure and control the stimulation device to apply the stimulation; and
a power source to provide power signals.

3. The apparatus of claim 2, wherein the footwear item further comprises a communication device to communicate with a mobile computing device, wherein the mobile computing device includes an application to track activity of the user and the processing device to receive and analyze the measured pressure to control the stimulation device.

4. The apparatus of claim 3, wherein the mobile computing device receives analytics from a network site based on the measured pressure and the stimulation to adjust the stimulation applied by the stimulation device.

5. The apparatus of claim 2, further comprising an accelerometer to provide acceleration measurements to the processing device for determining the one or more portions of the foot stimulated by the stimulation device.

6. The apparatus of claim 2, further comprising a ski with one or more bindings, wherein the processing device generates a signal to release the bindings in response to the measured pressure indicating one or more from a group of lateral and twisting motions.

7. The apparatus of claim 1, wherein the footwear item comprises a sock, and the stimulation device comprises one of an electrical stimulation device to provide electrical signals to the one or more portions of the foot and a vibration module to vibrate the one or more portions of the foot.

8. A method of providing corrective feedback using a footwear item, the method comprising:
measuring, via one or more pressure sensors disposed on a footwear item, pressure applied by a user; and
applying stimulation to one or more portions of a foot, via a stimulation device, to inform the user to alter pressure applied to those portions of the food to adjust one or more from a group of gait and a posture of a user.

9. The method of claim 8, further comprising:
analyzing the measured pressure via a processing device of the footwear item, and controlling the stimulation device to apply the stimulation.

10. The method of claim 9, wherein the footwear item further comprises a communication device to communicate with a mobile computing device including the processing device, and the method further comprises tracking, via the mobile computing device, activity of the user and receiving and analyzing the measured pressure to control the stimulation device.

11. The method of claim 10, further comprising:
receiving analytics at the mobile computing device from a network site based on the measured pressure and the stimulation; and
adjusting the stimulation applied by the stimulation device.

12. The computer-implemented method of claim 9, wherein the footwear item further comprises an accelerometer, and the method further comprises determining, via the processing device, the one or more portions of the foot stimulated by the stimulation device based on acceleration measurements.

13. The method of claim 9, wherein the footwear item comprises a ski with one or more bindings, and the method further comprises generating a signal, via the processing device, to release the bindings in response to the measured pressure indicating one or more from a group of lateral and twisting motions.

14. The method of claim 9, wherein the footwear item comprises a sock, and the stimulation device comprises one of an electrical stimulation device to provide electrical signals to the one or more portions of the foot and a vibration module to vibrate the one or more portions of the foot.
